(19) United States
(12) Patent Application Publication
(10) Pub. No.: US 2022/0160787 A1
(43) Pub. Date: May 26, 2022

(54) PLACENTA DERIVED ADHERENT CELLS FOR IMPROVED XENOPLANTATION

(71) Applicant: Celularity Inc., Florham Park, NJ (US)

(72) Inventors: QIAN YE, Martinsville, NJ (US); Joseph GLEASON, Point Pleasant, NJ (US); Lin KANG, Basking Ridge, NJ (US); Robert J. HARIRI, Bernardsville, NJ (US); Shuyang HE, Martinsville, NJ (US); Xiaokui ZHANG, Martinsville, NJ (US); Valentina ROUSSEVA, Florham Park, NJ (US)

(73) Assignee: Celularity Inc., Florham Park, NJ (US)

(21) Appl. No.: 17/439,671

(22) PCT Filed: Mar. 16, 2020
(86) PCT No.: PCT/US2020/023006

§ 371 Date: Sep. 15, 2021

Related U.S. Application Data
(60) Provisional application No. 62/819,154, filed on Mar. 15, 2019.

(51) Int. Cl. A61K 35/50

(52) U.S. Cl. CPC A61K 35/50; A61K 2035/122

(57) ABSTRACT

The present invention provides an isolated non-human placenta derived stem cell, wherein the stem cell expresses CD90 or wherein the stem cell expresses CD29. The present invention also provides a composition comprising an isolated non-human placenta derived stem cell of the invention or a population of isolated non-human placenta derived stem cells of the invention, and a carrier. The present invention also provides a composition of the invention for use in the manufacture of a medicament to reduce the incidence of rejection in a patient receiving a xenotransplant form a non-human donor. The present invention provides a method of treating a subject receiving a xenotransplant or xenotransfusion comprising the step of administering to the patient an effective amount of a non-human placenta derived stem cells.

### FIELD OF THE INVENTION

The present invention is directed to methods of improving xenotransfusions and xenotransplantations, e.g., by improving the clinical outcome thereof.

### BACKGROUND OF THE INVENTION

Xenotransplantation is one solution to overcome the major limitations between organ need and organ availability in the field of transplantation. To enable successful xenotransplantation, strategies have been developed to modulate T cell and B cell responses to solid organ and cellular xenografts, to manage xenograft rejection by components of the innate immune system, and to induce tolerance across the xenogeneic barrier. Despite these strategies, problems remain that prevent the widespread use of xenotransplanted tissues and organs. The present invention is directed to solving these and other problems.

Placenta derived stem cells (PDAC) are mesenchymal like stem cells that have been shown to be immunomodulatory, e.g., to have immunosuppressive qualities. Herein, non-human PDAC are isolated and described.

### SUMMARY OF THE INVENTION

The present invention provides an isolated non-human placenta derived stem cell, wherein the stem cell expresses CD90.

The present invention also provides a population of isolated non-human placenta derived stem cells, wherein the stem cell expresses CD29.

The present invention further provides a composition comprising an isolated non-human placenta derived stem cell of the invention or a population of isolated non-human placenta derived stem cells of the invention, and a carrier.

The present invention also provides a composition of the invention for use in the manufacture of a medicament to reduce the incidence of rejection in a patient receiving a xenotransplant form a non-human donor.

The present invention provides a method of treating a subject receiving a xenotransplant or xenotransfusion comprising the step of administering to the patient an effective amount of a non-human placenta derived stem cells.

### Terminology

As used herein, the term “isolated stem cell” means a stem cell that is substantially separated from other, non-stem cells of the tissue, e.g., placenta, from which the stem cell is derived. A stem cell is “isolated” if at least 50%, 60%, 70%, 80%, 90%, 95%, or at least 99% of the non-stem cells with which the stem cell is naturally associated are removed from the stem cell, e.g., during collection and/or culture of the stem cell.

As used herein, the term “isolated population of cells” means a population of cells that is substantially separated from other cells of the tissue, e.g., placenta, from which the population of cells is derived. A population of, e.g., stem cells is “isolated” if at least 50%, 60%, 70%, 80%, 90%, 95%, or at least 99% of the cells with which the population of stem cells are naturally associated are removed from the population of stem cells, e.g., during collection and/or culture of the population of stem cells.

As used herein, the term “placental stem cell” refers to a stem cell or progenitor cell that is derived from a mammalian placenta, regardless of morphology, cell surface markers, or the number of passages after a primary culture, which adheres to a tissue culture substrate (e.g., tissue culture plastic or a fibronectin-coated tissue culture plate). The term “placenta stem cell” as used herein does not, however, refer to a trophoblast, a cytotrophoblast, embryonic germ call, or embryonic stem cell, as those cells are understood by persons of skill in the art. A cell is considered a “stem cell” if the cell retains at least one attribute of a stem cell, e.g., a marker or gene expression profile associated with one or more types of stem cells; the ability to replicate at least 10-40 times in culture; multipotency, e.g., the ability to differentiate, either in vitro, in vivo or both, into cells of one or more of the three germ layers; the lack of adult (i.e., differentiated) cell characteristics, or the like. The terms “placental stem cell” and “placenta-derived stem cell” may be used interchangeably. Unless otherwise noted herein, the term “placental” includes the umbilical cord. The placental stem cells disclosed herein are, in certain embodiments, multipotent in vitro (that is, the cells differentiate in vitro under differentiating conditions), multipotent in vivo (that is, the cells differentiate in vivo), or both.

As used herein, “positive” or “+”, when used to indicate the presence of a particular cellular marker, means that the cellular marker is detectably present in fluorescence activated cell sorting over an isotype control; or is detectable above background in quantitative or semi-quantitative RT-PCR.

As used herein, “negative” or “−”, when used to indicate the presence of a particular cellular marker, means that the cellular marker is not detectably present in fluorescence activated cell sorting over an isotype control; or is not detectable above background in quantitative or semi-quantitative RT-PCR.

As used herein, “immunomodulation” and “immunomodulatory” mean causing, or having the capacity to cause, a detectable change in an immune response, and the ability to cause a detectable change in an immune response.

As used herein, “immunosuppression” and “immunosuppressive” mean causing, or having the capacity to cause, a detectable reduction in an immune response, and the ability to cause a detectable suppression of an immune response.

### BRIEF DESCRIPTION OF THE FIGURES

FIG. 1 shows representative images of sPDAC-B at passage 6 (left) and passage 10 (right) under light microscopy (200×).

FIG. 2 shows immunophenotyping of sPDAC-B by flow-cytometry: Passage 6 sPDAC-B cells were stained with anti-CD34 (FITC), anti-CD45 (PE), anti-CD44 (PE), anti-CD90 (PE), anti-CD29 (APC) and anti-CD105 (APC) monoclonal antibodies and analyzed with flow cytometry. Anti-IgG (PE, FITC, APC) antibodies were used as control for gating.

FIG. 3 shows immunophenotyping of sPDAC-A by flow-cytometry: Passage 6 sPDAC-A cells were stained with anti-CD34 (FITC), anti-CD45 (PE), anti-CD44 (PE), anti-CD90 (PE), anti-CD29 (APC) and anti-CD105 (APC) monoclonal antibodies and analyzed with flow cytometry. Anti-IgG (PE, FITC, APC) antibodies were used as control for gating.

FIG. 4 shows that sPDAC-B differentiate into adipocytes. Oil-droplets laden cells are present in the cell culture after 2-weeks of adipogenic induction (Left: transmission light). These oil-droplets took up oil-staining dye (Right).

FIG. 5 shows that sPDAC-B produce significantly high level of PGE-2 post stimulation of IL-1β. PGE2 was quantified with an ELISA kit (Cat# KGE004B, R&D Systems).

FIG. 6 shows that sPDAC-B inhibited the proliferation of human T cells in vitro. In a T cell proliferation assay, 1×10e5/100 uL CF SE-labeled human T cells was co-cultured with 1×10e5 sPDAC-B. Assay was performed in triplicate.

FIG. 7 shows expansion of sPDAC-B during continuous cell culture.

### DETAILED DESCRIPTION OF THE INVENTION

The present invention provides methods of treating a viral infection in a subject, comprising administering to the subject an amount of a composition comprising a plurality of placenta derived natural killer cells, effective to treat the viral infection in the subject.

The present invention provides an isolated non-human placenta derived stem cell, the stem cell expresses CD90. In some embodiments the isolated non-human placenta derived stem cell of the invention, said stem cell further expresses CD29. In some embodiments the isolated non-human placenta derived stem cell of the invention, said stem cell does not express CD34. In some embodiments the isolated non-human placenta derived stem cell of the invention, said stem cell does not express CD45. In some embodiments the isolated non-human placenta derived stem cell of the invention, said stem cell further expresses CD105. In some embodiments the isolated non-human placenta derived stem cell of the invention, said stem cell further expresses CD44.

In some specific embodiments the isolated non-human placenta derived stem cell of the invention, which is CD34−, CD45−, CD44−, CD90+ CD29+, and CD105+. In other specific embodiments the isolated non-human placenta derived stem cell of the invention, which is CD34−, CD45−, CD44−, CD90+ CD29+, and CD105−. In other specific embodiments the isolated non-human placenta derived stem cell of the invention, which is CD34−, CD45−, CD44+, CD90+ CD29+, and CD105+. In other specific embodiments the isolated non-human placenta derived stem cell of the invention, which is CD34−, CD45−, CD44+, CD90+ CD29+, and CD105−.

In some embodiments the isolated non-human placenta derived stem cell of the invention, which is a mesenchymal-like stem cell. In some embodiments the isolated non-human placenta derived stem cell of the invention, which is tissue culture plastic adhesive stem cell.

In some embodiments the isolated non-human placenta derived stem cell of the invention, is a porcine non-human placenta derived stem cell.

The present invention provides a population of isolated non-human placenta derived stem cells, the stem cell expresses CD90. In some embodiments the population of isolated non-human placenta derived stem cells of the invention, said stem cell further expresses CD29. In some embodiments the population of isolated non-human placenta derived stem cells of the invention, said stem cell does not express CD34. In some embodiments the population of isolated non-human placenta derived stem cells of the invention, said stem cell does not express CD45. In some embodiments the population of isolated non-human placenta derived stem cells of the invention, said stem cell further expresses CD105. In some embodiments the population of isolated non-human placenta derived stem cells of the invention, said stem cell further expresses CD44.

In some embodiments the population of isolated non-human placenta derived stem cells of the invention, which is CD34−, CD45−, CD44−, CD90+ CD29+, and CD105+. In some specific embodiments the population of isolated non-human placenta derived stem cells of the invention, which is CD34−, CD45−, CD44−, CD90+ CD29+, and CD105−. In other specific embodiments the population of isolated non-human placenta derived stem cells of the invention, which is CD34−, CD45−, CD44+, CD90+ CD29+, and CD105+. In other specific embodiments the population of isolated non-human placenta derived stem cells of the invention, which is CD34−, CD45−, CD44+, CD90+ CD29+, and CD105−.

In some embodiments the population of isolated non-human placenta derived stem cells of the invention, which is a mesenchymal-like stem cell. In some embodiments the population of isolated non-human placenta derived stem cells of the invention, which is tissue culture plastic adhesive stem cell.

In some embodiments the population of isolated non-human placenta derived stem cells of the invention, are porcine non-human placenta derived stem cells.

The present invention also provides a composition comprising an isolated non-human placenta derived stem cell of the invention or a population of isolated non-human placenta derived stem cells of the invention, and a carrier.

The present invention also provides a composition of the invention for use in the manufacture of a medicament to reduce the incidence of rejection in a patient receiving a xenotransplant form a non-human donor. In some embodiments the composition of the invention is provided for use in the manufacture of a medicament to reduce the incidence of graft versus host disease in a patient receiving a xenotransplant form a non-human donor. In some embodiments the composition of the invention is provided for use in the manufacture of a medicament to reduce the incidence of medical complications in a patient receiving a xenotransplant form a non-human donor. In some embodiments the composition for use according to the invention, the non-human donor is a pig. In preferred embodiments, the non-human donor is a genetically modified pig. In some embodiments the composition for use according to the invention, the genetically modified pig has been genetically modified to reduce the incidence of transplant rejection in a patient receiving tissue from said pig.

The present invention also provides a composition of the invention for use the treatment of tissue rejection in a patient receiving a xenotransplant form a non-human donor. In some embodiments the composition of the invention for use the treatment of graft versus host disease is provided for use in a patient receiving a xenotransplant form a non-human donor. In some embodiments the composition of the invention for use the treatment of complications is provided for use in a patient receiving a xenotransplant form a non-human donor. In some embodiments the composition for use according to the invention, the non-human donor is a pig. In preferred embodiments, the non-human donor is a genetically modified pig. In some embodiments the composition for use according to the invention, the genetically modified pig has been genetically modified to reduce the incidence of transplant rejection in a patient receiving tissue from said pig.

The present invention provides a method of treating a subject receiving a xenotransplant or xenotransfusion comprising the step of administering to the patient an effective amount of a non-human placenta derived stem cells.

In some embodiments of the invention, treating the subject comprises reducing the incidence of complications associated with said xenotransplant or xenotransfusion. In some embodiments of the invention, treating the subject comprises reducing the incidence of rejection associated with said xenotransplant or xenotransfusion. In some embodiments of the invention, treating the subject comprises reducing the incidence of graft versus host disease associated with said xenotransplant or xenotransfusion.

In some embodiments of the invention, said xenotransplant or xenotransfusion is from a primate. In preferred embodiments of the invention, said xenotransplant or xenotransfusion is from a baboon. In some embodiments of the invention, said xenotransplant or xenotransfusion is from a cow. In some embodiments of the invention, said xenotransplant or xenotransfusion is from a dog. In some embodiments of the invention, said xenotransplant or xenotransfusion is from a pig. In preferred embodiments of the invention, said pig is a genetically modified pig. In more preferred embodiments, said genetically modified pig has been genetically modified to reduce the incidence of transplant rejection in a patient receiving tissue from said pig.

In some embodiments of the invention, said administration is intravenous. In some embodiments of the invention, said administration is a local administration. In some embodiments of the invention, said administration occurs prior to said xenotransplant or xenotransfusion.

In some embodiments of the invention, said administration occurs concurrently with said xenotransplant or xenotransfusion. In some embodiments the method of the invention, said administration occurs subsequent to said xenotransplant or xenotransfusion. In some embodiments of the invention, said administration is a prophylactic measure to prevent complications associated with said xenotransplant or xenotransfusion. In some embodiments of the invention, said administration is in order to treat an occurrence of complications associated with said xenotransplant or xenotransfusion.

### Pharmaceutical Compositions

Immunosuppressive populations of placental stem cells, or populations of cells comprising placental stem cells, can be formulated into pharmaceutical compositions for use in vivo. Such pharmaceutical compositions comprise a population of placental stem cells, or a population of cells comprising placental stem cells, in a pharmaceutically acceptable carrier, e.g., a saline solution or other accepted physiologically acceptable solution for in vivo administration. Pharmaceutical compositions provided herein can comprise any of the placental stem cell populations, or placental stem cell types, described elsewhere herein. The pharmaceutical compositions can comprise fetal, maternal, or both fetal and maternal placental stem cells. The pharmaceutical compositions provided herein can further comprise placental stem cells obtained from a single individual or placenta, or from a plurality of individuals or placentae.

The pharmaceutical compositions provided herein can comprise any immunosuppressive number of placental stem cells. For example, a single unit dose of placental stem cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10₁₀, 5×10¹⁰, 1×10₁₁ or more placental stem cells.

The pharmaceutical compositions provided herein can comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, and the like.

### Administration

Administration of an isolated population of non-human PDAC cells or a pharmaceutical composition thereof may be systemic or local. In specific embodiments, administration is parenteral. In specific embodiments, administration of an isolated population of non-human PDAC cells or a pharmaceutical composition thereof to a subject is by injection, infusion, intravenous (IV) administration, intrafemoral administration, or intratumor administration. In specific embodiments, administration of an isolated population non-human PDAC cells or a pharmaceutical composition thereof to a subject is performed with a device, a matrix, or a scaffold. In specific embodiments, administration an isolated population of non-human PDAC cells or a pharmaceutical composition thereof to a subject is by injection. In specific embodiments, administration an isolated population of non-human PDAC cells or a pharmaceutical composition thereof to a subject is via a catheter. In specific embodiments, the injection of non-human PDAC cells is local injection. In more specific embodiments, the local injection is directly into a site which has received, is receiving, or will receive transplant of tissue from a non-human organism of the same species as the non-human PDAC cells. In specific embodiments, administration of an isolated population of non-human PDAC cells or a pharmaceutical composition thereof to a subject is by injection by syringe. In specific embodiments, administration of an isolated population of non-human PDAC cells or a pharmaceutical composition thereof to a subject is via guided delivery. In specific embodiments, administration of an isolated population of non-human PDAC cells or a pharmaceutical composition thereof to a subject by injection is aided by laparoscopy, endoscopy, ultrasound, computed tomography, magnetic resonance, or radiology.

Such cells can be administered to such an individual by absolute numbers of cells, e.g., said individual can be administered at about, at least about, or at most about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ non-human PDAC cells produced using the methods described herein. In other embodiments, non-human PDAC cells produced using the methods described herein can be administered to such an individual by relative numbers of cells, e.g., said individual can be administered at about, at least about, or at most about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ non-human PDAC cells produced using the methods described herein per kilogram of the individual. In other embodiments, non-human PDAC cells produced using the methods described herein can be administered to such an individual by relative numbers of cells, e.g., said individual can be administered at about, at least about, or at most about, 1×10⁵, 5×10⁵, 1×10⁶, 5×10⁶, 1×10⁷, 5×10⁷, 1×10⁸, or 5×10⁸ NK cells and/or ILC3 cells produced using the methods described herein per kilogram of the individual.

### EXAMPLES

### Example 1: Improved Transplantation and Xenotransplantation Using Placenta Derived Adherent Cells

To overcome the main immunologic barriers in xenotransplantation, strategies to induce immunotolerance across the xenogeneic barrier are desired to enable the development of clinical xenotransplantation protocols. To demonstrate feasibility, it is rationed that swine PDAC (sPDAC) could induce immunotolerance for Xenotransplantation between swine and primate (e.g. Baboon). In the invention described herein, we evaluate the potentials of placenta derived adherence cells (PDAC) in inducing immunotolerance to enable successful xenotransplantation, e.g., the transplantation of pig organs into a human recipient. To demonstrate proof-of-concept, a counterpart of human PDAC cells was established in swine followed by assessment of the ability to induce immunotolerance.

The initial phase for this proof of concept is the establishing of PDAC equivalent lines from GM-pig placentas for further in vitro and in vivo studies of immunomodulatory properties. The three aims for this study are as follows:
Aim1: Generate swine PDAC utilizing current human PDAC establishment and culture methods.
Aim 2: Swine PDACs will be characterized with selected in vitro assays that have been used for PDA001.
Aim 3: Swine PDAC will be tested for its function in a selected pre-clinical animal model.

In this study, we report that a swine placenta adherent cell line (sPDAC-B) has been established from full term GM-pig placentas with the procedures used for establishing human PDAC. sPDAC-B has been shown to have similar morphology and immunophenotype (CD34−CD45−CD105+CD90+CD29+) like human PDAC. sPDAC-B can be differentiated into adipocytes, stimulated by IL-1β to produce high level of immune modulator molecule PGE-2, and can inhibit human T cell proliferation in vitro. sPDAC-B can be expanded to passage 17 without loss of proliferation potential or sign of senescence. It is estimated that passage 9-10 of sPDAC will be equivalent to human PDAC at passage 6 in total accumulative population doubling. An inventory of over 100 million sPDAC-B from passage 1 to passage 7 have been created. From passage 1 to passage 9, sPDAC can proliferate over 30,000-fold. This inventory can be used as working bank to expand cells for multiple pre-clinical studies.

### Example 2: Establishment of Swine PDAC (sPDAC) Lines

Two separate pig placenta harvests of GM-pig placentas were received. A total of 6 and 8 placentas were processed respectively of first and 2nd delivery by 2-3 scientists following a protocol based on human PDAC establishment. Briefly, 20 to 40 grams of placenta tissues was extensively washed with sterile PBS containing antibiotics, minced to small tissue peices with sterile scalpels and then digested with collagenase. The digested tissues then were washed, cells were pelleted and cultivated in PDA-001 medium (DMEM media used for human PDAC culture containing 2% FBS, human EGF and human PDGF-BB). Cell isolated from each placenta were cultivated in T-225 flasks with medium change every 2-3 days after initial seeding.

Among all pig placenta cultures, adherent cells were observed 24 hours after seeding without apparent contamination. However, after about 1 week of culture, several of the cultures started to show signs of bacteria growth (the media became murky) and were had to be terminated. Individual swine placenta cultures from the placenta harvests which were free of contamination and resulted in cell culture were selected for future study. These cells were harvested, as Passage 0, about one month later. All cell cultures in second placenta harvest were contaminated with the same observations. Bacterial contamination in human PDAC establishment was very rare, it is suspected that there was a contamination of antibiotic resistance bacteria from the original pig placenta source during harvest. Preventive methods or higher caution protocols need to be implemented for future establishment to mitigate this problem. Such protocols are currently under consideration and/or investigation.

Two sPDAC lines (sPDAC-A and sPDAC-B) were established and carried out in culture to further passages beyond passage 0 (P0). It was noted that sPDACs proliferated significantly slower than human PDAC in the same medium. When seeded cells at 3000 cells/cm2, it took about three weeks to reach 80% confluence for next passage (human PDACs take about 7-10 days). It is not clear if this is pertaining to sPDAC or the cytokines in the growth medium are from human source. Nevertheless, both cell lines were expanded to passage 6 for characterization assays (human PDAC clinical drug products PDA-001 and PDA-002 are harvested at passage 6).

### Example 3: Phenotypic Characterization of sPDAC Lines

### Morphology of sPDAC

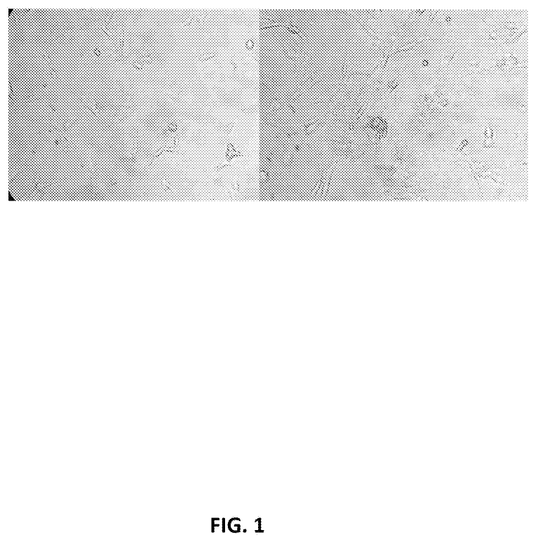

SPDAC lines were cultured and analyzed by light microscopy. FIG. 1 shows sPDAC-B at passage 6 and 10. Like human PDAC, sPDAC-B has a typical fibroblastic morphology. There were no significant morphological changes of sPDAC-B during different passages.

### Immunophenotype of sPDAC

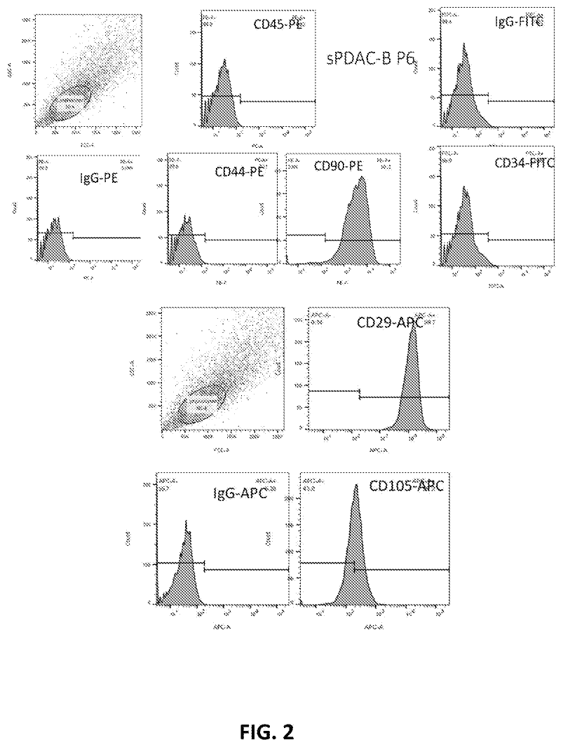

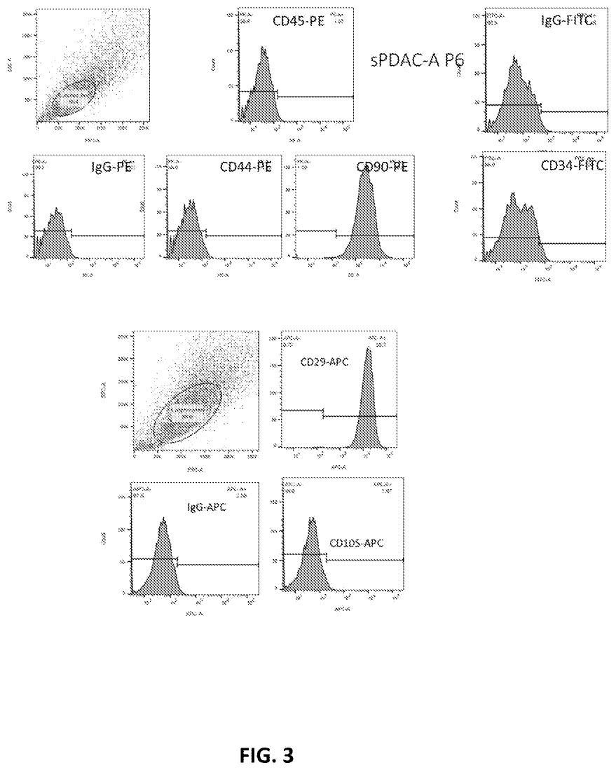

Human PDAC cells are negative for cell surface markers of CD34 and CD45 and positive for CD105, CD200, CD44, CD29, and CD90 (1, 2). Passage 6 sPDAC-A and sPDAC-B were analyzed with the following antibodies: anti-pig CD34 (goat-anti pig poly clonal, Cat# AF-3890-SP, BD Bioscience), anti-pig CD45 (Cat#MCA1222GA, BioRad), anti-CD90 (Cat#55596, BD Bioscience, recognize both human and pig), anti-CD44 (Cat#554478, BD Bioscience, recognize both human and pig), anti-CD29 (Cat#561496, BD Bioscience, recognize pig protein) Anti-human CD105 (Cat#561439, BD Bioscience). The data of FACS analysis of sPDAC-B is shown in FIG. 2, which is CD34−CD45−CD44−CD90+CD29+CD105+. This phenotype exhibited by sPDAC-B is similar to that of human PDAC and human mesenchymal stromal cells (MSC) except for CD44. sPDAC-A shows also positive for CD29 and CD90, but it is negative for CD105 (FIG. 3). CD105 is a key cell surface marker for both human and porcine MSC (3), suggesting that sPDAC-A may not be an MSC like cell line comparable to human PDAC. Based on the above phenotypic characterization and the phenotype of human PDAC, isolation of cells based on negativity for CD34 and/or CD45 and positivity for one or more of CD 90, CD29, CD105 and/or CD44 may be selected. Such selection can be readily performed by one of skill in the art, e.g., by phenotypic analysis of a clonal or polyclonal population, or by sorting (e.g., FACS or MACS based sorting) of a population.

### Example 4: Functional Characterization of sPDAC Lines

### Differentiation of sPDAC into Adipocytes

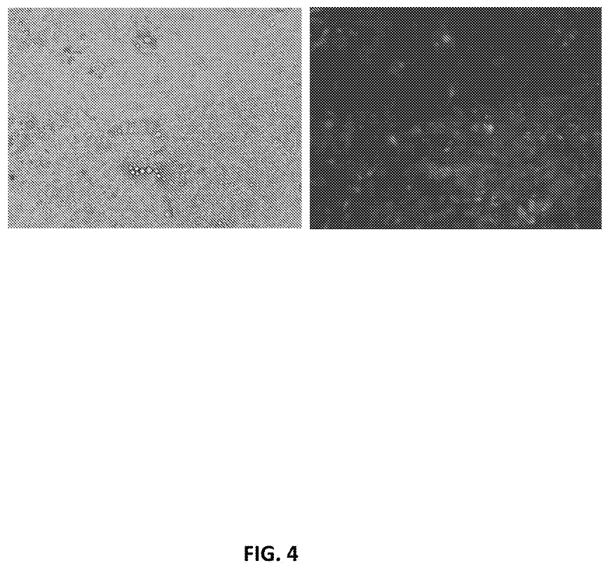

One of the key features of human PDAC and MSC cells is their ability to differentiate into specific cell lineages including adipocytes under defined induction conditions (1, 3). To examine if sPDAC-B can be differentiated into adipocytes, passage 6 sPDAC-A and sPDAC-B were plated in 6-well culture plate and adipogenic induction media (Stem Proadipogenic Kit, Cat#1007001, Thermo Fisher Scientific) was added when cells reached 90% confluency. Adipogenic induction medium was replenished every 2-3 days for 2 weeks. Accumulation of oil-droplets was evident in majority of sPDAC-B cells in the culture, these oil-droplets were shown (FIG. 4) to take up oil-droplet specific dye (HCS Liqid Tox Green Neutral Lipid, Cat#H34475, Thermo Fisher Scientific). However, sPDAC-A cells did not show any adipocyte differentiation potential in this assay (data not shown). These results confirm that sPDAC-B has differentiation potential like human PDAC while sPDAC-A does not.

### In Vitro Function of sPDAC B: Secretion of PGE-2 when Exposed to IL-1

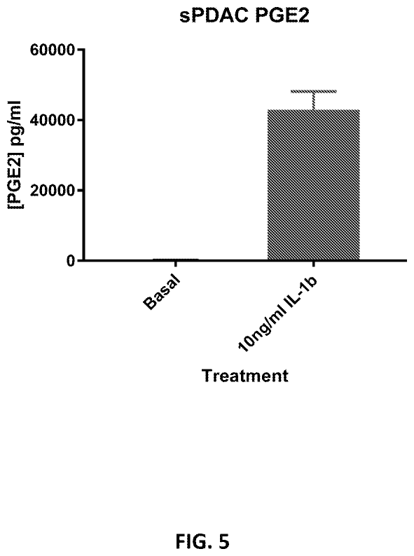

Prostaglandin E2 (PGE2) is a key molecule involved during inflammation. Human PDAC has been shown to secret high levels of PGE2 upon stimulation with interleukin 1β (IL-1β). To determine whether sPDAC-B has similar in vitro function, sPDAC-B (passage 6) were seeded at 1×10e5 cells/well in 6-well plate (triplicates) and treated with pig IL-1β (Cat#681-P1-010, R&D Systems) overnight. Supernatant were collected and PGE-2 were measured using an ELISA kit (FIG. 5). Supernatant from untreated cells (basal) were used as control.

### Inhibition of Human T Cell Proliferation by sPDAC

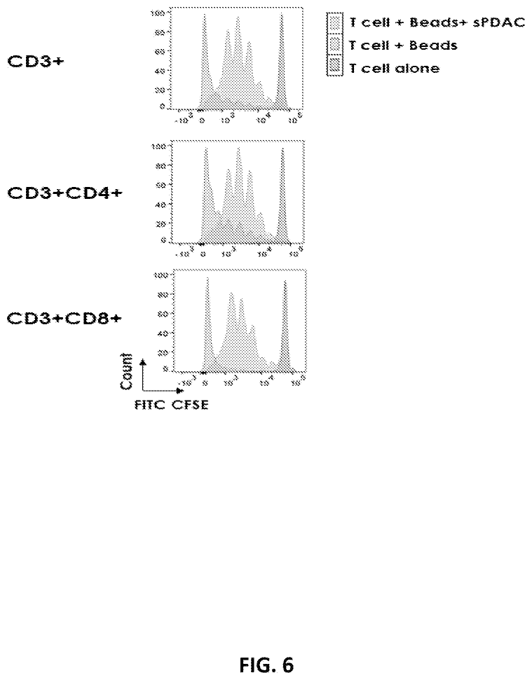

Human PDAC has immuno-modulation functions including inhibition of T cell proliferation in vitro (1). To examine if sPDAC-B has similar function, sPDAC-B (passage 6) were co-cultured with human T cells (labeled with CFSE dye) and stimulated with anti-CD3/CD28 beads. Labeled cells without bead treatment and cells treated only with beads were used as negative and positive control. FACS analysis was performed after 5 days to evaluate T cell proliferation. The data (FIG. 6) demonstrated that with beads stimulation, T cell (red) did not undergone any proliferation and at the presence of only anti-CD3/CD28 beads, majority of the cells proliferated and resulted in the shift of CFSE signal to base line (blue lines). At the presence of sPDAC-B, total T cells (CD3+) and CD4+ as well as CD8+ T cells showed significant delay of proliferation (yellow lines). These data showed that the proliferation of CD4+ and CD8+ cells was delayed/inhibited by the sPDAC-B. Therefore, sPDAC-B has demonstrated the similar in vitro immuno-modulation function as human PDAC.

### Proliferation of sPDAC-B Cells

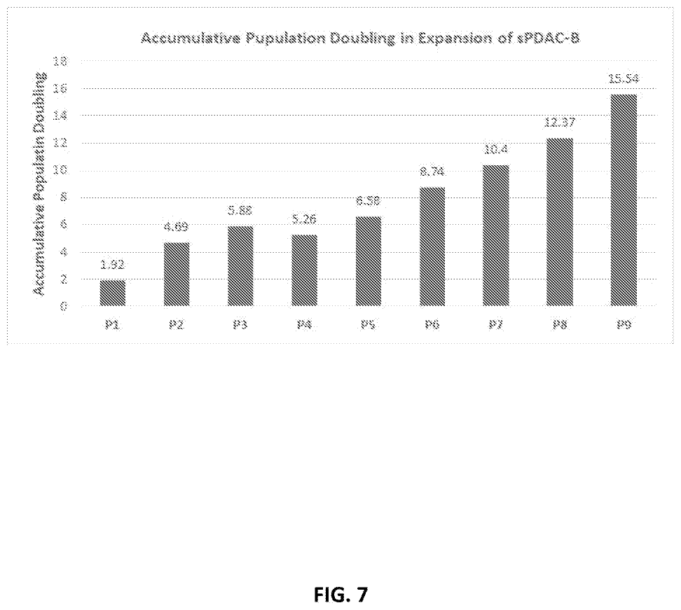

To evaluate the cell proliferation potential of sPDAC-B, P0 cells was thawed and continue culture in PDA-001 medium (establishing media) with 50% of media change every 2-3 days a week with standard cell culture protocol. Cells were seeded at 3000 cells/cm2 in T75 or T225 flasks and culture to reach about 70% cell confluence and harvested with trypsinization. Cells were also frozen at each passage to build an inventory of working cell bank for future studies. FIG. 7 shows the accumulative population of sPDAC-B from passage 1 to passage 9. As mentioned above, sPDAC-B appeared to grow significantly slower than human PDAC. It also appears to detach from flask when it reaches higher confluency. Therefore, sPDAC-B was harvested at relatively lower density (60-70%). Human PDACs can be harvested at higher density and resulted in higher accumulative population per passage. As a result, at passage 6, sPDAC-B has only undergone 9 population doublings comparing with human PDAC were undergone 15-20 population doublings (DPL). sPDAC-B reached 15 DPL at passage 9.

To further evaluate the proliferation potential of sPDAC-B, four different culture experiments were carried out for sPDAC-B and the accumulative fold of expansion is summarized in Table 1. It was observed that sPDAC-B can be expanded to at least passage 17 without any significant change of morphology and proliferation potential. During these cultures, an inventory of cryopreserved sPDAC-B has been established consisting over 100×10e6 cells from P1 to P7 (Table 2). This inventory can be used as working cell bank to expand cells for pre-clinical studies.

TABLE 1
Accumulative Fold of Expansion of sPDAC-B during culture

| Experiment-1 | | Experiment-2 | | Experiment-3 | | Experiment-4 | |
|---|---|---|---|---|---|---|---|
| P1 | 4 | P1 | 15 | P6 | 3 | P12 | 7 |
| P2 | 26 | P2 | 16 | P7 | 18 | P13 | 37 |
| P3 | 42 | P3 | 17 | P8 | 226 | P14 | 137 |
| P4 | 27 | P4 | 24 | P9 | 1286 | P15 | 533 |
| P5 | 68 | P5 | 88 | P10 | 12278 | P16 | 2045 |
| P6 | 283 | | | P11 | 59572 | P17 | 9817 |
| P7 | 898 | | | P12 | 297860 | | |
| P8 | 3529 | | | | | | |
| P9 | 31763 | | | | | | |

Note:
Experiments started from thawing vials s one passage earlier shown in each experiment in the table.

TABLE 2
Inventory of Cryopreserved sPDAC-B

| Passage | Vials | Cells (× 10e6) | |
|---|---|---|---|
| P1 | 11 | 9 | |
| P2 | 16 | 44 | |
| P3 | 17 | 17 | |
| P4 | 8 | 20 | |
| P5 | 5 | 5 | |
| P6 | 14 | 14 | Sum (P1 to P7) 1 × 10e6 |
| P7 | 9 | 23 | 132 |
| P8 | 13 | 13 | |
| P9 | 28 | 28 | |
| P10 | 34 | 34 | |

### Example 5: Conclusions and Recommendations

We have demonstrated the feasibility of establishing swine PDAC cells from full term GM-pig placentas. Despite some level of culture contamination, one sPDAC line (sPDAC-B) was established and characterized using different assays including phenotype, differentiation, PGE-2 secretion and inhibition of T cells proliferation. These assays demonstrate sPDAC-B is comparable to human PDAC in these biological features and activities. Further characterization including cytokine release, expanded immuno- phenotyping panel will depend on the study needs and availability of assay reagents.

Initially, sPDAC was planned to be expanded to passage-6 as human PDAC. However, it was found that sPDAC-B proliferated slower and accumulated significantly lower population doublings (about 8-9 PDL) at passage 6. In the case of human PDAC, it has 20 DPL (1). Therefore, we recommend that sPDAC-B at passage 9-10 (DPL of 15-18) should be more equivalent to human PDAC for future studies.

### Equivalents

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

### REFERENCES CITED

1. Liu et al. (2014) Human placenta-derived adherent cells induce tolerogenic immune responses. Clinical & Translational Immunology 2014; 3: e14; doi:10.1038/cti.2015.5.
2. Chen et al. (2015) Human placenta-derived adherent cells improve cardiac performance in mice with chronic heart failure. Stem Cell Translational Medicine. 4:269-275.
3. Noort et al. (2011) Human versus porcine mesenchymal stromal cells: phenotypes, differentiation potential, immunomodulation and cardiac improvement after transplantation. J. Cell Mol. Med. 16:1827-1839.

What is claimed is:

1. An isolated non-human placenta derived stem cell, wherein the stem cell expresses CD90.

2. The isolated non-human placenta derived stem cell of claim 1, wherein said stem cell further expresses CD29.

3. The isolated non-human placenta derived stem cell of claim 1 or claim 2, wherein said stem cell does not express CD34.

4. The isolated non-human placenta derived stem cell of any one of claims 1-3, wherein said stem cell does not express CD45.

5. The isolated non-human placenta derived stem cell of any one of claims 1-4, wherein said stem cell further expresses CD105.

6. The isolated non-human placenta derived stem cell of any one of claims 1-5, wherein said stem cell further expresses CD44.

7. The isolated non-human placenta derived stem cell of any one of claims 1-6, which is CD34−, CD45−, CD44−, CD90+ CD29+, and CD105+.

8. The isolated non-human placenta derived stem cell of any one of claims 1-6, which is CD34−, CD45−, CD44−, CD90+ CD29+, and CD105−.

9. The isolated non-human placenta derived stem cell of any one of claims 1-6, which is CD34−, CD45−, CD44+, CD90+ CD29+, and CD105+.

10. The isolated non-human placenta derived stem cell of any one of claims 1-6, which is CD34−, CD45−, CD44+, CD90+ CD29+, and CD105−.

11. The isolated non-human placenta derived stem cell of any one of claims 1-10, which is a mesenchymal-like stem cell.

12. The isolated non-human placenta derived stem cell of any one of claims 1-11, which is tissue culture plastic adhesive stem cell.

13. The isolated non-human placenta derived stem cell of any one of claims 1-12, wherein the non-human placenta derived stem cell is a porcine non-human placenta derived stem cell.

14. A population of isolated non-human placenta derived stem cells, wherein the stem cell expresses CD90.

15. The population of isolated non-human placenta derived stem cells of claim 14, wherein said stem cell further expresses CD29.

16. The population of isolated non-human placenta derived stem cells of claim 14 or claim 15, wherein said stem cell does not express CD34.

17. The population of isolated non-human placenta derived stem cells of any one of claims 14-16, wherein said stem cell does not express CD45.

18. The population of isolated non-human placenta derived stem cells of any one of claims 14-17, wherein said stem cell further expresses CD105.

19. The population of isolated non-human placenta derived stem cells of any one of claims 14-18, wherein said stem cell further expresses CD44.

20. The population of isolated non-human placenta derived stem cells of any one of claims 14-19, which is CD34−, CD45−, CD44−, CD90+ CD29+, and CD105+.

21. The population of isolated non-human placenta derived stem cells of any one of claims 14-19, which is CD34−, CD45−, CD44−, CD90+ CD29+, and CD105−.

22. The population of isolated non-human placenta derived stem cells of any one of claims 14-19, which is CD34−, CD45−, CD44+, CD90+ CD29+, and CD105+.

23. The population of isolated non-human placenta derived stem cells of any one of claims 14-19, which is CD34−, CD45−, CD44+, CD90+ CD29+, and CD105−.

24. The population of isolated non-human placenta derived stem cells of any one of claims 14-23, which is a mesenchymal-like stem cell.

25. The population of isolated non-human placenta derived stem cells of any one of claims 14-24, which is tissue culture plastic adhesive stem cell.

26. The population of isolated non-human placenta derived stem cells of any one of claims 14-25, wherein the non-human placenta derived stem cells are porcine non-human placenta derived stem cells.

27. A composition comprising an isolated non-human placenta derived stem cell of any one of claims 1-13 or a population of isolated non-human placenta derived stem cells of any one of claims 14-26, and a carrier.

28. The composition of claim 27 for use in the manufacture of a medicament to reduce the incidence of rejection in a patient receiving a xenotransplant form a pig.

29. The composition of claim 27 for use in the manufacture of a medicament to reduce the incidence of graft versus host disease in a patient receiving a xenotransplant form a pig.

30. The composition of claim 27 for use in the manufacture of a medicament to reduce the incidence of medical complications in a patient receiving a xenotransplant form a pig.

31. The composition for use according to any one of claims 28-30, wherein the pig is a genetically modified pig.

32. The composition for use according to claim 31, wherein the genetically modified pig has been genetically modified to reduce the incidence of transplant rejection in a patient receiving tissue from said pig.

33. The composition of claim 26 for use the treatment of tissue rejection in a patient receiving a xenotransplant form a pig.

34. The composition of claim 26 for use the treatment of graft versus host disease in a patient receiving a xenotransplant form a pig.

35. The composition of claim 26 for use the treatment of complications in a patient receiving a xenotransplant form a pig.

36. The composition for use according to any one of claims 32-35, wherein the pig is a genetically modified pig.

37. The composition for use according to claim 36, wherein said genetically modified pig has been genetically modified to reduce the incidence of transplant rejection in a patient receiving tissue from said pig.

38. A method of treating a subject receiving a xenotransplant or xenotransfusion comprising the step of administering to the patient an effective amount of a non-human placenta derived stem cells.

39. The method of claim 38, wherein treating the subject comprises reducing the incidence of complications associated with said xenotransplant or xenotransfusion.

40. The method of claim 38, wherein treating the subject comprises reducing the incidence of rejection associated with said xenotransplant or xenotransfusion.

41. The method of claim 38, wherein treating the subject comprises reducing the incidence of graft versus host disease associated with said xenotransplant or xenotransfusion.

42. The method of any one of claims 38-41, wherein said xenotransplant or xenotransfusion is from a primate.

43. The method of claim 42, wherein said xenotransplant or xenotransfusion is from a baboon.

44. The method of any one of claims 38-41, wherein said xenotransplant or xenotransfusion is from a cow.

45. The method of any one of claims 38-41, wherein said xenotransplant or xenotransfusion is from a dog.

46. The method of any one of claims 38-41, wherein said xenotransplant or xenotransfusion is from a pig.

47. The method of any one of claims 38-46, wherein said pig is a genetically modified pig.

48. The method of claim 47, wherein said genetically modified pig has been genetically modified to reduce the incidence of transplant rejection in a patient receiving tissue from said pig.

49. The method of any one of claims 38-48, wherein said administration is intravenous.

50. The method of any one of claims 38-48, wherein said administration is a local administration.

51. The method of any one of claims 38-50, wherein said administration occurs prior to said xenotransplant or xenotransfusion.

52. The method of any one of claims 38-50, wherein said administration occurs concurrently with said xenotransplant or xenotransfusion.

53. The method of any one of claims 38-50, wherein said administration occurs subsequent to said xenotransplant or xenotransfusion.

54. The method of any one of claims 38-53, wherein said administration is a prophylactic measure to prevent complications associated with said xenotransplant or xenotransfusion.
The method of any one of claims 38-53, wherein said administration is in order to treat an occurrence of complications associated with said xenotransplant or xenotransfusion.
